(19) United States
(12) Patent Application Publication
(10) Pub. No.: US 2011/0095233 A1
(43) Pub. Date: Apr. 28, 2011

(54) Production of Synthesis Gas

(76) Inventors: Diane Hildebrandt, Johannesburg (ZA); David Glasser, Johannesburg (ZA); Brendon Patrick Hausberger, North Riding (ZA)

(21) Appl. No.: 11/886,854

(22) PCT Filed: Mar. 23, 2006
(86) PCT No.: PCT/IB2006/000651

§ 371 Date: Mar. 5, 2008

(30) Foreign Application Priority Data
Mar. 23, 2005 (ZA) 20047676

(51) Int. Cl. C01B 3/34; C07C 1/02; B01D 53/34

(52) U.S. Cl. 252/373; 422/168

(57) ABSTRACT

A method of producing Synthesis Gas is provided which includes combining the product of a coal gasification reaction with the product of a methane reforming reaction. The coal gasification and methane reforming reactions can take place either in the same vessel or in separate vessels.

### FIELD OF THE INVENTION

This invention relates to a method for the production of synthesis gas

### BACKGROUND TO THE INVENTION

“Synthesis Gas”, or “Syngas”, is a mixture of carbon monoxide (CO) and hydrogen (H₂), typically when produced in the molar ratio CO:H₂ of 1:3 or 1:1.

Currently Synthesis Gas is made by one of two processes, either from coal by gasification with oxygen, usually from air, and water, or from methane by reforming with oxygen (known as thermal reforming or partial oxidative reforming) or water (known as steam reforming).

The chemical reactions associated with the formation of Synthesis Gas from carbon, usually from coal, are as follows:

C+H₂O→CO+H₂(gasification)

The CO:H₂ ratio produced in this way may not be correct for use in downstream synthesis processes. The water gas shift reaction as described below is thus used to adjust the CO:H₂ ratio.

CO+H₂O→CO₂+H₂

This adjustment results in the emission of CO₂ or water depending on the required adjustment.

Similarly for methane, Syngas can be produced by the following reaction:

CH₄+H₂O→CO+3H₂(reforming)

The CO:H₂ ratio produced in this way may not be correct for use in downstream synthesis processes. The water gas shift reaction as described above is used to adjust the CO:H₂ ratio. This adjustment also results in the emission of CO₂ or water depending on the required adjustment.

There are many reactors which are commercially used to produce Synthesis Gas. These include reactors based on the Lurgi process, the Winkler process and the Wellman process.

The Syngas produced is used, in turn, to produce a wide range of carbon based chemicals. For example, methanol and other hydrocarbon products are made from Synthesis Gas via various processes. Ammonia is another product synthesised from Synthesis Gas in reaction with the nitrogen in air.

The Syngas production processes are endothermic and, as a consequence, a considerable amount of energy is required to run these processes and, to meet these requirements, there are significant carbon dioxide emissions. It is noted that carbon dioxide is a major greenhouse gas, and hence its emission into the atmosphere is not environmentally friendly.

### OBJECT OF THE INVENTION

It is an object of this invention to provide a method of producing Synthesis Gas which will at least partially alleviate some of the abovementioned problems.

### SUMMARY OF THE INVENTION

In accordance with this invention there is provided a method of producing Synthesis Gas which includes combining the product of a coal gasification reaction with the product of a methane reforming reaction.

A further feature of the invention provides ratio of coal fed to the coal gasification reaction to natural gas fed to the methane reforming reaction to be varied to produce Synthesis Gas of predetermined composition.

According to one aspect of the invention there is provided for the coal gasification reaction to occur separately from the methane reforming reaction and for the products of the separate reactions to be combined.

Further according to this aspect of the invention there is provided for the product of at least the gasification reaction to have undesirable components removed therefrom prior to combining with the product of the methane reforming reaction; alternatively or in addition for undesirable components to be removed from the Synthesis Gas after production thereof.

According to a second aspect of the invention there is provided for the coal gasification reaction to occur in the same vessel as the methane reforming reaction.

Further according to this aspect of the invention there is provided for undesirable components to be removed from the Synthesis Gas after production thereof.

The invention also provides a Synthesis Gas production unit which includes a coal feed and a natural gas feed and wherein the unit subjects the coal feed to a gasification reaction and the natural gas to a methane reforming reaction.

A further feature of the invention provides for gasification reaction and reforming reaction to take place simultaneously.

According to one aspect of the invention there is provided for the gasification reaction to take place in a separate vessel to the reforming reaction.

Further according to this aspect of the invention there is provided for the unit to include means for combining the products of the gasification reaction and reforming reaction; and for the unit to include means for removing undesirable components from the product of the gasification reaction.

According to a second aspect of the invention there is provided for the gasification reaction to take place in the same vessel as the reforming reaction.

Further according to this aspect of the invention there is provided for the unit to include means for removing undesirable components from the Synthesis Gas produced.

### DESCRIPTION OF EMBODIMENTS OF THE INVENTION

While coal gasification processes have been widely used for the production of Synthesis Gas because of the cost benefits of using coal as the major starting material, these processes are, by their very nature, inefficient in energy consumption and produce large carbon dioxide emissions. According to the present invention the products of the methane reforming process is combined with that of the carbon gasification reaction. It has been found that combining the products of the methane reforming reaction and the carbon gasification reaction facilitates the more efficient use of coal and methane resources while reducing the environmental impact per ton of product produced.

The combination of the products of methane and coal reactions has been demonstrated to result in a synergistic effect, even with minor additions of methane to the process. This benefit has been found to result from the preferred heat of combustion of the methane combustion together with the capacity to adjust the carbon:hydrogen ratio. The benefits include:
reduced carbon dioxide emissions;
a reduction or elimination of the need for the water gas shift reaction;
the ability to reduce or balance energy requirements of the reactions and downstream processes;
increased process economies; and
a reduction in the equipment required.

These benefits result primarily from a reduction in carbon dioxide emissions and it is this aspect in particular that is a quite unexpected result of combining the products of the gasification and reforming reactions.

### Process Conditions:

Coal gasification operates in a temperature range of between 500 and 1200° C. and from atmospheric to 70 bars for a variety of commercial implementations. Any of these would be suitable for the application of separate gasification implementations. The gasification process can make use of pure oxygen or an enriched or standard air feed.

Standard methane reforming technologies operate in the temperature range between 700 to 1100° C. and make use of either oxygen (partial oxidation reformers) or steam and oxygen (steam reformers).

The ratio of coal to natural gas used would be varied as required to produce Synthesis Gas of predetermined composition. This greatly assists in economising downstream processing of the Synthesis Gas as it permits the Synthesis Gas to be produced with a desired composition. The temperature of the process can be adjusted to alter the hydrogen, carbon monoxide ratio by means of the water gas shift reaction.

### Reactions Occurring:

### Coal Gasification Reactions

The reactions below do not include the pyrolysis and drying reaction that would take place in the unit.

C+½O₂→CO

C+O₂→CO₂

H₂+O₂→H₂O

These reactions are exothermic and produce the energy required by the endothermic reduction, pyrolysis and cracking reactions.

### Reduction Reactions

C+CO₂→2CO

C+H₂O→CO+H₂

Other reactions know to take place in the coal gasifier include:

### Water Gas Shift

CO+H₂O [symbol] CO₂+H₂

### Methane Formation

Exothermic Methane Forming Reactions

C+2H₂→CH₄

CO+3H₂→CH₄+H₂O
Endothermic Methane Forming Reactions

3C+2H₂O→CH₄+2CO

### Cracking Reactions

4CₙHₘ→mCH₄+(4n−m)C

### Hydrogenation

2CₙHₘ+(4n−m)H₂→2nCH₄
Hydrogenation:

CₙHₘ+(2n−m/2)H₂→nCH₄

### Methane Reformer Reactions

Combustion of Methane to provide the required process energy:

CH₄+O₂→CO+2H₂O

CH₄+2O₂→CO₂+2H₂O

### Reforming Reactions

CH₄+O₂→CO+2H₂

CH₄+H₂O→CO+3H₂

In addition, the water gas shift reaction described above is known to occur in the methane reformers.

The net effect of these reactions is exothermic thus obviating the need to add energy to the process. This results in a significant energy saving over the known individual processes.

### Process Examples:

The reforming and gasification reactions can be performed either in separate vessels or in a single vessel. While there are scale economic savings to be achieved in the inclusion of these reactions into a single vessel, there are process costs. The product stream from coal gasification is generally far richer in sulphide products and other undesirable components than the methane reformer product. It is usually necessary to remove these components prior to any further processing of the Synthesis Gas to avoid the poisoning of the downstream catalyst beds. Due to the differences in the gasification and product streams, there are advantages to both the merging of the processes and the separation. Where the gas purification technology is more suited to concentrated toxin removal, the separation of coal gasification and methane reforming and the separate cleaning of these streams prior to merging may be desirable. A simple flow diagram of such a process is shown in FIG. 1.

It will be understood that the reforming reaction and gasification reaction could take place simultaneously or at different times to each other.

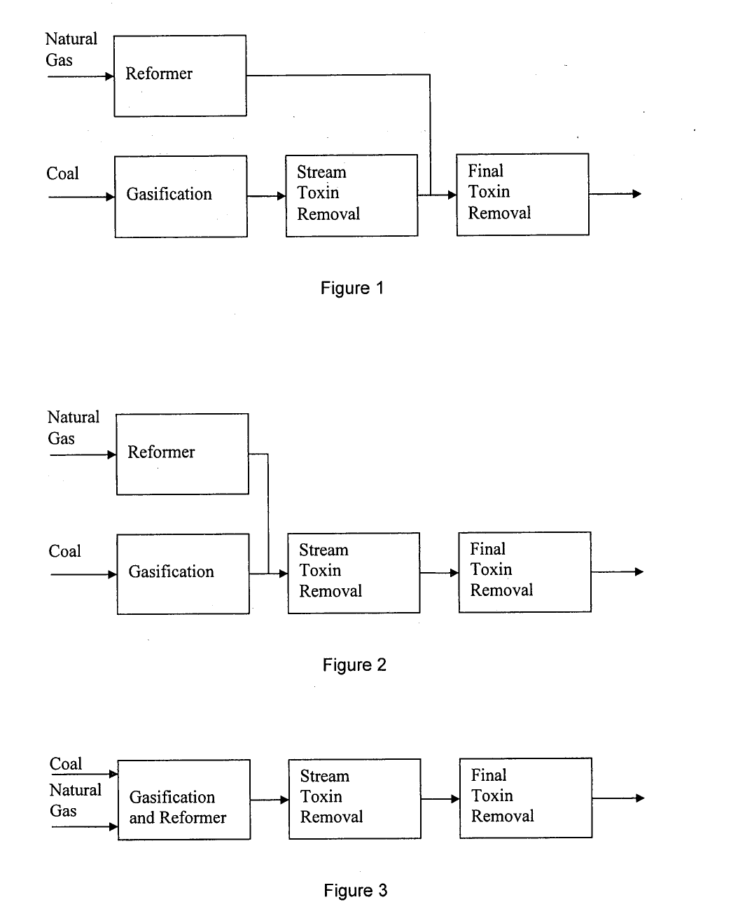

Alternatively, where a gas purification technology is selected that would work preferentially with a lower toxin composition, the integration of the reformer and gasifier reactions would lower the toxins to the point where the technology can be applied, as shown in the flow diagram in FIG. 2.

Both of these process combinations would permit the production of any desired carbon monoxide ratio while making the optimal use of the gas purification technology.

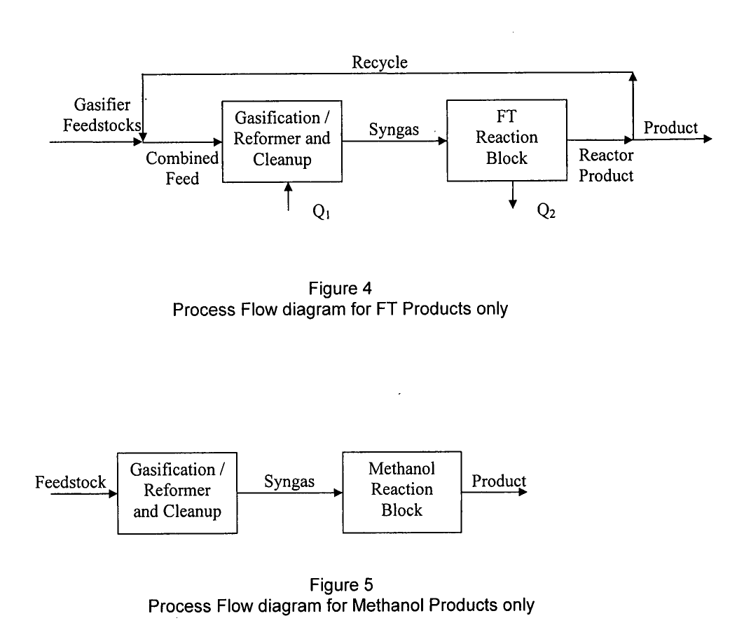

A third option, which requires the development of appropriate technology, permits the gasification of the coal and the reforming of the methane in the same vessel. A simple flow diagram of this process is given in FIG. 3.

The above examples illustrate a further aspect of the invention, namely a unit for the production of Synthesis Gas. The unit is characterised in that it includes a coal feed and a natural gas feed and in that it subjects the coal feed to a gasification reaction and the natural gas to a methane reforming reaction. These reactions can take place in separate vessels in the unit, as illustrated with reference to FIGS. 1 and 2, or in a single vessel, as illustrated with reference to FIG. 3.

Where the reactions occur in separate vessels the unit will include means for combining the products of the reactions. It will also include means for removing undesirable components from either or both of the product streams. The unit will furthermore include means for varying the ratio of the coal feed to natural gas feed to enable Synthesis Gas of predetermined composition to be produced. This will enable the Synthesis Gas composition to be tailored to suit downstream processes.

### Downstream Processing

The downstream processes in which the Synthesis Gas can be used are many. They include the production of hydrocarbons, such as methanol, and the production of nitrogen-hydrogen complexes such as ammonia. It has been found that integrating downstream processes to produce hydrocarbons or nitrogen-hydrogen complexes can generate even greater savings in energy consumption and carbon dioxide emission. These figures can be reduced to almost zero by the optimum combination or integration of processes. Thus, the energy from exothermic processes is used to supply endothermic processes in an energy neutral situation. Likewise, carbon dioxide emitted from one process is recycled to other processes, thus minimising or eliminating any excess carbon dioxide which has to be released into the atmosphere.

Integrating processes to make the Synthesis Gas with those to produce nitrogen-hydrogen complexes such as ammonia have the further advantage that both the major components of air, oxygen and nitrogen, are used in the integrated processes.

The following examples illustrate the possible scenarios of downstream processes. In the examples “Gasification/Reformer and Cleanup” refers to the “Reformer”, “Gasification”, “Stream Toxin Removal” and “Final Toxin Removal” steps in FIGS. 1 to 3.

### EXAMPLES

All of the report mass balances are based on a feed of 100 mol/s of coal (simplified to its carbon content only) and can be scaled on this basis. The processes are designed to theoretically require only feed product interchange. This does not permit for the heat exchange inefficiencies.

### Fischer Tropsch (“FT”) Type Products Only

These flow rates are indicative only and do not include the usual contaminants of a gasification and reformer process, namely H₂S and mercury amongst others.

This simplified flow sheet also disregards the toxin removal steps as these are established and licensed technologies.

For the molar and mass balance shown in Tables 1 and 2, the product is characterised as an octane stream, this is representative of a product distribution corresponding to an alpha of between 0.86 and 0.88. For an alpha of between 0.84 and 0.86 (representative product Heptane) one would use the values in Tables 4 and 5, and for a value of between 0.81 and 0.84 one could use hexane as the representative product stream and obtain the values in Tables 7 and 8. The enthalpy flows are given in tables 2, 5 and 8 respectively. The unit energy loads are given in tables 3, 6 and 9.

The flows shown do not represent the actual vessel output flows but rather the system net flows from unit blocks to be expected in the process. This is necessary as the gross flows would depend on the specifics of the catalyst selection and vessel design. These detailed design parameters would influence the degree of water gas shift equilibrium as well as the product alkane/oxygenate distribution. The purpose of these examples is to provide a demonstration of the general theory of the present invention.

TABLE 1
Molar balance for an Alpha of between 0.86 and 0.88

| | Gasifier | Combined | | Reactor | | Re- |
|---|---|---|---|---|---|---|
| mol/s | Feedstocks | Feed | Syngas | Product | Product | cycle |
| Coal | 100.00 | 100.00 | 0.00 | 0.00 | 0.00 | 0.00 |
| Methane | 30.21 | 30.21 | 0.00 | 0.00 | 0.00 | 0.00 |
| Water | 48.57 | 48.57 | 0.00 | 0.00 | 0.00 | 0.00 |
| Oxygen | 9.04 | 9.04 | 0.00 | 0.00 | 0.00 | 0.00 |
| Carbon | 0.00 | 0.00 | 193.77 | 0.00 | 0.00 | 0.00 |
| Monoxide | | | | | | |
| Carbon | 0.00 | 63.56 | 0.00 | 96.88 | 33.33 | 63.56 |
| Dioxide | | | | | | |
| Hydrogen | 0.00 | 0.00 | 108.99 | 0.00 | 0.00 | 0.00 |
| Octane | 0.00 | 0.00 | 0.00 | 12.11 | 12.11 | 0.00 |

TABLE 2
Mass balance for an Alpha of between 0.86 and 0.88

| | Gasifier | | | Reactor | | |
|---|---|---|---|---|---|---|
| g/s | Feedstocks | Combined Feed | Syngas | Product | Product | Recycle |
| Coal | 1200.00 | 1200.00 | 0.00 | 0.00 | 0.00 | 0.00 |
| Methane | 483.38 | 483.38 | 0.00 | 0.00 | 0.00 | 0.00 |
| Water | 874.29 | 874.29 | 0.00 | 0.00 | 0.00 | 0.00 |
| Oxygen | 289.34 | 289.34 | 0.00 | 0.00 | 0.00 | 0.00 |
| Carbon | 0.00 | 0.00 | 5425.47 | 0.00 | 0.00 | 0.00 |
| Monoxide | | | | | | |
| Carbon | 0.00 | 2796.45 | 0.00 | 4262.87 | 1466.42 | 2796.45 |
| Dioxide | | | | | | |
| Hydrogen | 0.00 | 0.00 | 217.99 | 0.00 | 0.00 | 0.00 |
| Octane | 0.00 | 0.00 | 0.00 | 1380.59 | 1380.59 | 0.00 |
| Total | 2847.01 | 5643.46 | 5643.46 | 5643.46 | 2847.01 | 2796.45 |
| Enthalpy | −16152 | −41178 | −21434 | −41178 | −16152 | −25026 |
| [kJ/s] | | | | | | |

TABLE 3
Unit Energy Loads for an Alpha of between 0.86 and 0.88

| Unit | Energy Flow [kJ/s] |
|---|---|
| Gasification/Reformer and Cleanup Q₁ | 19743.77 |
| FT Reaction Block Q₂ | 19743.77 |

TABLE 4
Molar balance for an Alpha of between 0.84 and 0.86

| | Gasifier | Combined | | Reactor | | Re- |
|---|---|---|---|---|---|---|
| mol/s | Feedstocks | Feed | Syngas | Product | Product | cycle |
| Coal | 100.00 | 100.00 | 0.00 | 0.00 | 0.00 | 0.00 |
| Methane | 31.53 | 31.53 | 0.00 | 0.00 | 0.00 | 0.00 |
| Water | 48.92 | 48.92 | 0.00 | 0.00 | 0.00 | 0.00 |
| Oxygen | 9.08 | 9.08 | 0.00 | 0.00 | 0.00 | 0.00 |
| Carbon | 0.00 | 0.00 | 195.98 | 0.00 | 0.00 | 0.00 |
| Monoxide | | | | | | |
| Carbon | 0.00 | 64.44 | 0.00 | 97.99 | 33.54 | 64.44 |
| Dioxide | | | | | | |
| Hydrogen | 0.00 | 0.00 | 111.99 | 0.00 | 0.00 | 0.00 |
| Heptane | 0.00 | 0.00 | 0.00 | 14.00 | 14.00 | 0.00 |

TABLE 5
Mass balance for an Alpha of between 0.84 and 0.86

| | Gasifier | | | Reactor | | |
|---|---|---|---|---|---|---|
| g/s | Feedstocks | Combined Feed | Syngas | Product | Product | Recycle |
| Coal | 1200.00 | 1200.00 | 0.00 | 0.00 | 0.00 | 0.00 |
| Methane | 504.51 | 504.51 | 0.00 | 0.00 | 0.00 | 0.00 |
| Water | 880.60 | 880.60 | 0.00 | 0.00 | 0.00 | 0.00 |
| Oxygen | 290.65 | 290.65 | 0.00 | 0.00 | 0.00 | 0.00 |
| Carbon | 0.00 | 0.00 | 5487.32 | 0.00 | 0.00 | 0.00 |
| Monoxide | | | | | | |
| Carbon | 0.00 | 2835.54 | 0.00 | 4311.47 | 1475.93 | 2835.54 |
| Dioxide | | | | | | |
| Hydrogen | 0.00 | 0.00 | 223.97 | 0.00 | 0.00 | 0.00 |
| Heptane | 0.00 | 0.00 | 0.00 | 1399.83 | 1399.83 | 0.00 |
| Total | 2875.76 | 5711.30 | 5711.30 | 5711.30 | 2875.76 | 2835.54 |
| Enthalpy | −16351 | −41727 | −21679 | −41727 | −16351 | −25376 |
| [kJ/s] | | | | | | |

TABLE 6
Unit Energy Loads for an Alpha of between 0.84 and 0.86

| Unit | Energy Flow [kJ/s] |
|---|---|
| Gasification/Reformer and Cleanup Q₁ | 20048.40 |
| FT Reaction Block Q₂ | 20048.40 |

TABLE 7
Molar balance for an Alpha of between 0.81 and 0.84

| | Gasifier | Combined | | Reactor | | Re- |
|---|---|---|---|---|---|---|
| mol/s | Feedstocks | Feed | Syngas | Product | Product | cycle |
| Coal | 100.00 | 100.00 | 0.00 | 0.00 | 0.00 | 0.00 |
| Methane | 33.31 | 33.31 | 0.00 | 0.00 | 0.00 | 0.00 |
| Water | 49.42 | 49.42 | 0.00 | 0.00 | 0.00 | 0.00 |
| Oxygen | 9.14 | 9.14 | 0.00 | 0.00 | 0.00 | 0.00 |
| Carbon | 0.00 | 0.00 | 198.92 | 0.00 | 0.00 | 0.00 |
| Monoxide | | | | | | |
| Carbon | 0.00 | 65.61 | 0.00 | 99.46 | 33.85 | 65.61 |
| Dioxide | | | | | | |
| Hydrogen | 0.00 | 0.00 | 116.03 | 0.00 | 0.00 | 0.00 |
| Hexane | 0.00 | 0.00 | 0.00 | 16.58 | 16.58 | 0.00 |

TABLE 8
Mass balance for an Alpha of between 0.81 and 0.84

| | Gasifier | | | Reactor | | |
|---|---|---|---|---|---|---|
| g/s | Feedstocks | Combined Feed | Syngas | Product | Product | Recycle |
| Coal | 1200.00 | 1200.00 | 0.00 | 0.00 | 0.00 | 0.00 |
| Methane | 532.92 | 532.92 | 0.00 | 0.00 | 0.00 | 0.00 |
| Water | 889.55 | 889.55 | 0.00 | 0.00 | 0.00 | 0.00 |
| Oxygen | 292.47 | 292.47 | 0.00 | 0.00 | 0.00 | 0.00 |
| Carbon | 0.00 | 0.00 | 5569.67 | 0.00 | 0.00 | 0.00 |
| Monoxide | | | | | | |
| Carbon | 0.00 | 2886.80 | 0.00 | 4376.17 | 1489.37 | 2886.80 |
| Dioxide | | | | | | |
| Hydrogen | 0.00 | 0.00 | 232.07 | 0.00 | 0.00 | 0.00 |
| Hexane | 0.00 | 0.00 | 0.00 | 1425.57 | 1425.57 | 0.00 |
| Total | 2914.94 | 5801.74 | 5801.74 | 5801.74 | 2914.94 | 2886.80 |
| Enthalpy | −16626 | −42461 | −22004 | −42461 | −16626 | −25835 |
| [kJ/s] | | | | | | |

TABLE 9
Unit Energy Loads for an Alpha of between 0.86 and 0.88

| Unit | Energy Flow [kJ/s] |
|---|---|
| Gasification/Reformer and Cleanup Q₁ | 20456.93 |
| FT Reaction Block Q₂ | 20456.93 |

The theoretical carbon dioxide limits of conventional processes are as follows:

TABLE 10

| | Ratio Tons Coal:Tons |
|---|---|
| Tons CO₂/Ton Hydrocarbons | Natural Gas in Feed |
| 0.75 | 0.5 |
| 1.6 | 1.5 |
| 2.5 | 4 |
| 3.7 | Coal Feed |

Comparing the theoretical carbon dioxide production in Table 10 with that produced in the processes according to the invention it will be noted that a great reduction in carbon dioxide emissions results from the processes of the invention.

For purposes of further comparison, Tables 1A, 2A and 3A below illustrate molar flows where a coal only feed is used according to prior art processes.

TABLE 1A
Molar balance for an Alpha of between 0.86 and 0.88
Coal Only Feed

| | Gasifier | Combined | | Reactor | | Re- |
|---|---|---|---|---|---|---|
| Mol/s | Feedstocks | Feed | Syngas | Product | Product | cycle |
| Coal | 100.00 | 100.00 | 0.00 | 0.00 | 0.00 | 0.00 |
| Water | 64.74 | 64.74 | 0.00 | 0.00 | 0.00 | 0.00 |
| Oxygen | 10.08 | 10.08 | 0.00 | 0.00 | 0.00 | 0.00 |
| Carbon | 0.00 | 0.00 | 115.10 | 0.00 | 0.00 | 0.00 |
| Monoxide | | | | | | |
| Carbon | 0.00 | 15.10 | 0.00 | 57.55 | 42.45 | 15.10 |
| Dioxide | | | | | | |
| Hydrogen | 0.00 | 0.00 | 64.74 | 0.00 | 0.00 | 0.00 |
| Octane | 0.00 | 0.00 | 0.00 | 7.19 | 7.19 | 0.00 |

TABLE 2A
Mass balance for an Alpha of between 0.86 and 0.88
Coal Only Feed

| | | Com- | | | | |
|---|---|---|---|---|---|---|
| | Gasifier | bined | | Reactor | | |
| g/s | Feedstocks | Feed | Syngas | Product | Product | Recycle |
| Coal | 1200.00 | 1200.00 | 0.00 | 0.00 | 0.00 | 0.00 |
| Water | 1165.37 | 1165.37 | 0.00 | 0.00 | 0.00 | 0.00 |
| Oxygen | 322.55 | 322.55 | 0.00 | 0.00 | 0.00 | 0.00 |
| Carbon | 0.00 | 0.00 | 3222.74 | 0.00 | 0.00 | 0.00 |
| Monoxide | | | | | | |
| Carbon | 0.00 | 664.31 | 0.00 | 2532.15 | 1867.85 | 664.31 |
| Dioxide | | | | | | |
| Hydrogen | 0.00 | 0.00 | 129.49 | 0.00 | 0.00 | 0.00 |
| Octane | 0.00 | 0.00 | 0.00 | 820.07 | 820.07 | 0.00 |
| Total | 2687.92 | 3352.23 | 3352.23 | 3352.23 | 2687.92 | 664.31 |

TABLE 3A
Unit Energy Loads for an Alpha of between 0.86 and 0.88
Coal Only Feed

| | Energy Flow [kJ/s] |
|---|---|
| Flow into Gasifier Unit | 11727.84 |
| Flow out of Reactor Unit | 11727.84 |

Comparing the figures in the Tables 1A, 2A and 3A to those in Tables 1, 2 and 3 illustrates that it is possible to reduce the carbon dioxide emissions by approximately half per molar unit of product produced by using the process of the present invention. This is both highly beneficial and unexpected.

### Methanol Product Only

The flows shown below do not represent the actual reactor output flows but rather the system net flows from unit blocks to be expected in the process. This is necessary as the gross flows would depend on the specifics of the catalyst selection and reactor design.

TABLE 11
Molar Balance on Methanol Process

| | Gasifier | | Reactor |
|---|---|---|---|
| mol/s | Feedstocks | Raw Syngas | Product |
| Coal | 100.00 | 0.00 | 0.00 |
| Methane | 32.89 | 0.00 | 0.00 |
| Water | 123.16 | 0.00 | 0.00 |
| Oxygen | 24.07 | 0.00 | 0.00 |
| Carbon | 0.00 | 94.47 | 0.00 |
| Monoxide | | | |
| Carbon Dioxide | 0.00 | 38.42 | 38.42 |
| Hydrogen | 0.00 | 188.94 | 0.00 |
| Methanol | 0.00 | 0.00 | 94.47 |

TABLE 12
Mass Balance on Methanol Process

| | Gasifier | | Reactor |
|---|---|---|---|
| g/s | Feedstocks | Raw Syngas | Product |
| Coal | 1200.00 | 0.00 | 0.00 |
| Methane | 526.19 | 0.00 | 0.00 |
| Water | 2216.96 | 0.00 | 0.00 |
| Oxygen | 770.24 | 0.00 | 0.00 |
| Carbon | 0.00 | 2645.13 | 0.00 |
| Monoxide | | | |
| Carbon Dioxide | 0.00 | 1690.38 | 1690.38 |
| Hydrogen | 0.00 | 377.88 | 0.00 |
| Methanol | 0.00 | 0.00 | 3023.01 |
| Total | 4713.39 | 4713.39 | 4713.39 |
| Enthalpy [kJ/s] | −37684.07284 | −25577.92072 | −37684.07284 |

TABLE 13
Unit Energy Loads for Methanol Process

| Unit | Energy Flow [kJ/s] |
|---|---|
| Gasification/Reformer and Cleanup Q₁ | 12106.15 |
| Methanol Reaction Block Q₂ | 12106.15 |

### Combined Process

For a combination of process products there are two distinct process possibilities.

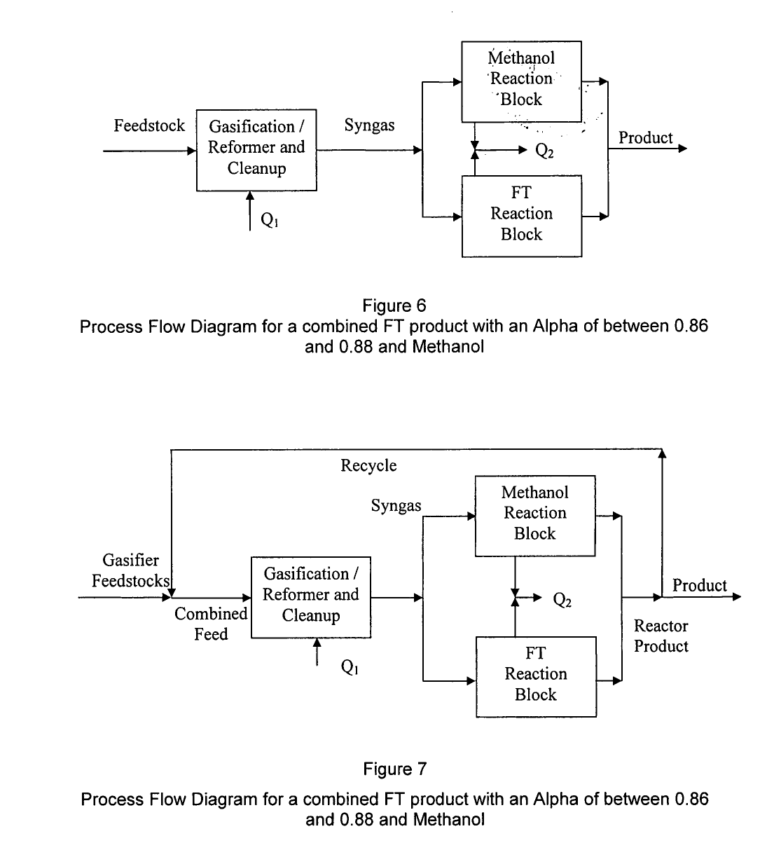

For product with less than 4.56 moles of Octane equivalent (alpha=0.86-0.88) per 100 moles of coal fed, the process diagram is given below as FIG. 6.

The corresponding molar, mass and enthalpy flows are given in Tables 14, 15 and 16.

TABLE 14
Molar balance for a combined FT product with an Alpha
of between 0.86 and 0.88 and Methanol

| | Gasifier | | |
|---|---|---|---|
| Mol/s | Feedstocks | Syngas | Product |
| Coal | 100.00 | 0.00 | 0.00 |
| Methane | 31.88 | 0.00 | 0.00 |
| Water | 95.06 | 0.00 | 0.00 |
| Oxygen | 18.41 | 0.00 | 0.00 |
| Carbon | 0.00 | 131.88 | 0.00 |
| Monoxide | | | |
| Carbon | 0.00 | 0.00 | 36.50 |
| Dioxide | | | |
| Hydrogen | 0.00 | 158.82 | 0.00 |
| Nitrogen | 0.00 | 0.00 | 0.00 |
| Octane | 0.00 | 0.00 | 4.56 |
| Methanol | 0.00 | 0.00 | 58.88 |

TABLE 15
Mass balance for a combined FT product with an Alpha
of between 0.86 and 0.88 and Methanol

| | Gasifier | | |
|---|---|---|---|
| g/s | Feedstocks | Syngas | Product |
| Coal | 1200.00 | 0.00 | 0.00 |
| Methane | 510.06 | 0.00 | 0.00 |
| Water | 1711.12 | 0.00 | 0.00 |
| Oxygen | 589.07 | 0.00 | 0.00 |
| Carbon | 0.00 | 3692.60 | 0.00 |
| Monoxide | | | |
| Carbon | 0.00 | 0.00 | 1606.01 |
| Dioxide | | | |
| Hydrogen | 0.00 | 317.64 | 0.00 |
| Nitrogen | 0.00 | 0.00 | 0.00 |
| Octane | 0.00 | 0.00 | 520.13 |
| Methanol | 0.00 | 0.00 | 1884.11 |
| Total | 4010.24 | 4010.24 | 4010.24 |
| Enthalpy | −29572 | −14588 | −29572 |
| [kJ/s] | | | |

TABLE 16
Unit Energy Loads for Methanol Process

| Unit | Energy Flow [kJ/s] |
|---|---|
| Gasification/Reformer and Cleanup Q₁ | 14983.57 |
| Reaction Blocks Q₂ | 14983.57 |

For product with greater than 4.56 moles of Octane equivalent (alpha=0.86-0.88) per 100 moles of coal fed, the process diagram is given below as FIG. 7.

The corresponding molar, mass and enthalpy flows are given in Tables 17, 18 and 19.

Similar demonstrations can be shown for other alpha value as this will affect the composition of the feedstock and operation of the units.

TABLE 17
Molar balance for a combined FT product with an Alpha of
between 0.86 and 0.88 and Methanol

| | Gasifier | Combined | | Reactor | | Re- |
|---|---|---|---|---|---|---|
| Mol/s | Feedstocks | Feed | Syngas | Product | Product | cycle |
| Coal | 100.00 | 100.00 | 0.00 | 0.00 | 0.00 | 0.00 |
| Methane | 31.01 | 31.01 | 0.00 | 0.00 | 0.00 | 0.00 |
| Water | 70.95 | 70.95 | 0.00 | 0.00 | 0.00 | 0.00 |
| Oxygen | 13.55 | 13.55 | 0.00 | 0.00 | 0.00 | 0.00 |
| Carbon | 0.00 | 0.00 | 163.98 | 0.00 | 0.00 | 0.00 |
| Monoxide | | | | | | |
| Carbon | 0.00 | 32.96 | 0.00 | 67.82 | 34.85 | 32.96 |
| Dioxide | | | | | | |
| Hydrogen | 0.00 | 0.00 | 132.98 | 0.00 | 0.00 | 0.00 |
| Nitrogen | 0.00 | 0.00 | 0.00 | 0.00 | 0.00 | 0.00 |
| Octane | 0.00 | 0.00 | 0.00 | 8.48 | 8.48 | 0.00 |
| Methanol | 0.00 | 0.00 | 0.00 | 28.34 | 28.34 | 0.00 |

TABLE 18
Mass balance for a combined FT product with an Alpha of between
0.86 and 0.88 and Methanol

| | Gasifier | | | Reactor | | |
|---|---|---|---|---|---|---|
| g/s | Feedstocks | Combined Feed | Syngas | Product | Product | Recycle |
| Coal | 1200.00 | 1200.00 | 0.00 | 0.00 | 0.00 | 0.00 |
| Methane | 496.22 | 496.22 | 0.00 | 0.00 | 0.00 | 0.00 |
| Water | 1277.09 | 1277.09 | 0.00 | 0.00 | 0.00 | 0.00 |
| Oxygen | 433.61 | 433.61 | 0.00 | 0.00 | 0.00 | 0.00 |
| Carbon | 0.00 | 0.00 | 4591.37 | 0.00 | 0.00 | 0.00 |
| Monoxide | | | | | | |
| Carbon | 0.00 | 1450.40 | 0.00 | 2984.01 | 1533.61 | 1450.40 |
| Dioxide | | | | | | |
| Hydrogen | 0.00 | 0.00 | 265.95 | 0.00 | 0.00 | 0.00 |
| Nitrogen | 0.00 | 0.00 | 0.00 | 0.00 | 0.00 | 0.00 |
| Octane | 0.00 | 0.00 | 0.00 | 966.41 | 966.41 | 0.00 |
| Methanol | 0.00 | 0.00 | 0.00 | 906.90 | 906.90 | 0.00 |
| Total | 3406.92 | 4857.32 | 4857.32 | 4857.32 | 3406.92 | 1450.40 |
| Enthalpy | −22612 | −35592 | −18139 | −35592 | −22612 | −12980 |
| [kJ/s] | | | | | | |

TABLE 19
Unit Energy Loads for Methanol Process

| Unit | Energy Flow [kJ/s] |
|---|---|
| Gasification/Reformer and Cleanup Q₁ | 17452.48 |
| Reaction Blocks Q₂ | 17452.48 |

It will be appreciated that the process conditions will be varied to take into account different feed materials and also to control the composition of the Synthesis Gas and products obtained therefrom.

It will further be apparent from the above examples that combining the products of the gasification reaction and reforming reaction result in a significant reduction in carbon dioxide emissions. This not only has great environmental benefits but also results in much greater process economies than can be achieved through conventional processes.

1. A process of producing Synthesis Gas comprising the steps of:
gasifying coal by means of a coal gasification reaction,
feeding natural gas into a methane reformer where it is subject to a methane reforming reaction including both a methane steam reaction and an autothermal/partial oxidative reforming reaction, and
combining the product of the coal gasification reaction with the product of the methane reforming reaction to produce the Synthesis Gas,
wherein the ratio of coal gasified to natural gas fed to the methane reforming reaction is adjusted to produce Synthesis Gas of selected predetermined composition and to minimize resulting carbon dioxide emissions.

2. (canceled)

3. The process according to claim 1 wherein the coal gasification reaction occurs separately from the methane reforming reaction and the products of the separate reactions are combined.

4. The process according to claim 3 wherein the product of at least the coal gasification reaction is fed through a gas scrubber to remove undesirable components therefrom prior to combining with the product of the methane reforming reaction.

5. The process according to claim 3 wherein the Synthesis Gas is fed through a gas scrubber to remove undesirable components therefrom after production of the Synthesis Gas.

6. The process according to claim 1 wherein the coal gasification reaction occurs in the same vessel as the methane reforming reaction.

7. The process according to claim 6 wherein the Synthesis Gas is fed through a gas scrubber to remove undesirable components therefrom after production of the Synthesis Gas.

8. A Synthesis Gas production unit comprising
a coal feed into a coal gasifier where the coal is gasified,
a natural gas feed into a methane reformer where methane is reformed, and
a collector in which the product of the coal gasification reaction with the product of the methane reforming reaction are combined to produce the Synthesis Gas.

9. The Synthesis Gas production unit as claimed in claim 8, wherein the gasification reaction and reforming reaction take place simultaneously.

10. The Synthesis Gas production unit as claimed in claim 8 in which the coal gasifier and the methane reformer are separated from each other.

11. (canceled)

12. The Synthesis Gas production unit as claimed in claim 9, wherein a gas scrubber is connected to receive the product of the gasification reaction to remove undesirable components therefrom prior to combining it with the product of the methane reforming reaction.

13. The Synthesis Gas production unit as claimed in claim 8 in which the gasification reaction takes place in the same vessel as the reforming reaction.

14. The Synthesis Gas production unit as claimed in claim 13 which includes a gas scrubber which is connected to receive the Synthesis Gas from the collector and remove undesirable components therefrom.
